(19) United States
(12) Patent Application Publication
(10) Pub. No.: US 2012/0088234 A1
(43) Pub. Date: Apr. 12, 2012

(54) DEVICE FOR PERFORMING PCRS

(76) Inventors: Gerhard Hartwich, Munchen (DE); Norbert Persike, Munich (DE)

(21) Appl. No.: 13/127,964

(22) PCT Filed: May 5, 2010
(86) PCT No.: PCT/DE10/75120

§ 371 Date: Dec. 13, 2011

(30) Foreign Application Priority Data
Nov. 5, 2009 (DE) 10 2009 044 431.9

(51) Int. Cl. C12Q 1/68; C12M 1/40

(52) U.S. Cl. 435/6.11; 435/289.1; 435/287.2; 435/6.12

(57) ABSTRACT

A device is described to perform reactions, in which the device includes at least one test cell with a cavity to receive the test sample and at least a first, a second and a third spatially discrete regulatable temperature units. The three temperature units thus define three spatially discrete temperature areas. At least one means is provided to perform a rotary movement of the test cell. The cavity provided to receive the test sample can be moved across the three spatially discrete temperature areas, in which the cavity in one position of the test cell, remains in contact with a least two of the temperature areas.

### FIELD OF THE INVENTION

The present invention is directed to a device for performing polymerase chain reactions (PCRs).

### BACKGROUND OF THE INVENTION

Polymerase chain reaction (PCR) is one of the basic methods used in molecular biology for multiplying DNA molecules. It enables the direct verification of the smallest quantities of DNA or RNA. In recent years this method has been used and mastered in many laboratories, because it provides a broad range of applications and may be used at several levels. PCR is used in almost every field of science and medicine, including forensic medicine, prenatal diagnostics, oncology and, last but not least, in microbiological diagnostics. For example, in the field of clinical diagnostics, PCR is generally the method of choice, e.g., for confirming pathogenic agents in a sample. Equally, it is used in the food industry as a detection method for confirming pathogenic germs.

PCR involves an enzyme reaction to amplify nucleic acid molecules amounts, wherein the reaction occurring in a very small volume of an aqueous or liquid reaction mix. A sample containing nucleic acid is mixed together with primers, nucleotides and a polymerase, which are also necessary for the reaction. By addition of buffers and preferably bivalent ions such as Mg²⁺ to the reaction mix, the optimum reaction conditions are obtained for the relevant kind of application.

PCR is based on a cycle of three steps that each operates at a different temperature, namely, a denaturation step, a hybridization step and an extension step.

During denaturation the reaction mix is heated to a temperature of greater than 90° C., preferably from 94° C. to 95° C. By this step, complementary strands of the double-stranded DNA separate, i.e., the DNA is “melted” or “denatured”, and is present as single strands. The denaturation is a very fast process and usually occurs within seconds.

During hybridization (also called “annealing”) the temperature is set at a so-called “annealing temperature”. At this temperate the primers bind to the DNA, i.e., they hybridize with the DNA. The “annealing temperature” is dependent on the length and sequence of the primers and can be specifically defined for each primer based on these features. As a rule the “annealing temperatures” lie in a temperature range of between 55° C. and 65° C., but for specific applications can also be set lower or higher. The hybridization does not require much time and usually takes place within seconds.

For extension, that is the next step after “annealing”, the temperature is raised once more, preferably to 70° C. to 74° C. This is the ideal working temperature for the polymerase enzyme usually used, which adds further nucleotides to the DNA strands being synthesized starting from the bound primers. At the stated temperature range, loose connections between the primers and template DNA particles that are not completely complementary, break free once again.

The extension step is that step of PCR, which generally takes the most time. So, for example, the working and reaction speed of the polymerase is the time-limiting step. This means that the shorter the time period is at the optimal temperature for extension, e.g., about 70° C. to 74° C., the shorter the newly synthesized DNA strands will be. Generally several seconds suffice in the extension step (15 to 60 seconds), but in standard PCR procedures a period of time in the region of minutes (from one to several minutes) is selected for DNA extension if very large DNA molecules have to be synthesized.

At each repetition of the three steps above, the number of the copied DNA molecules is doubled. In this way, after 20 cycles about one million molecules may be synthesized from a single DNA double strand.

The number of cycles may be selected in accordance with the type of application, the nature of the assay and the specific demands and requirements of the reaction. Accordingly, the time period settings can be adapted to suit the specific requirements of given types of applications.

Essentially, the details set out above apply to standard PCR procedures in which copies of an existing double stranded test DNA are made. There is however, an entire range of special PCR procedures, which must be adapted to the requirements of individual cases. An example of a highly specialized PCR application is the so-called RT-PCR (reverse transcriptase PCR). In this case, with the help of a reverse transcriptase enzyme, amplification is obtained from test RNA. As a first step, a hybrid double stranded RNA/DNA is created, the reverse transcriptase synthesizing a complementary DNA strand to the existing test RNA. In a second step, namely a “general” PCR protocol the newly synthesized DNA-strand can be used after denaturing the RNA/DNA hybrid as a template.

It is worth mentioning in this context that a still further type of PCR exists: Real-Time-PCR. This quantitative PCR method enjoys favored status in molecular biology laboratories, since it permits the detection of test DNA and the amplification products in real time and, at the same time, the analysis of the quantity of test DNA.

The analysis of DNA quantities at the end of a PCR does not permit direct conclusions about the number of molecules originally present, because at the start and at the end of the PCR the requirements for polymerase are not optimized and hence the amplification does not run evenly throughout the entire reaction time. Thus, the quantification at the end of a PCR can be very imprecise. Significantly more precise quantification is possible, if the number of synthesized DNA molecules is detected during the reaction, that is, after each individual cycle. This quantification as a rule takes place using fluorescent labelling of newly synthesized DNA molecules. The precise analysis of quantities of test DNA is essential, especially in medical diagnosis but also in seeking targets and basic research. This is why in these areas the option of real time quantification is very much sought after.

In conventional PCR applications the reaction mix including a nucleic acid containing sample (“template DNA”), primers, nucleotides, polymerase and buffer are mixed in a reaction vessel, wherein usually between 10 μl and 100 μl of reaction mix are placed in the reaction vessel. The reaction vessel is placed in a receiving unit and subjected to a number of temperature cycles. The receiving unit is usually made up of a metal block that can be set at stated temperatures, which are fitted with hollows wherein the reaction vessels can be placed. The reaction vessels, which show a volume capacity in a range of around 200 μl to 500 μl are usually available as single enclosable vessels or as a strip or plate with several hollows, known as multi-well strips or plates. The number and relative distances in multi-well plates or strips are designed for hollows in the temperature setting metal blocks, so that inserting them within the metal blocks is possible.

With all these conventional PCR applications applied devices the temperature cycles are usually generated by recurrent heating and cooling of the temperature settable blocks, whereupon the control is mostly performed using Peltier modules. A major disadvantage of these repeated heating and cooling stages is the time required for execution.

Significant differences exist between the heating and cooling rates of the temperature settable blocks according to supplier, quality and price class. As a rule of thumb for the heating stage, a rate of between approximately 1° C. and 10° C. per second can be assumed, and cooling rates are somewhat slower. In order to achieve the required temperature targets per cycle a period of time of between 15 seconds and 2 minutes is required, which with about 30 cycles for heating and cooling produces a required time of up to one hour. This time limitation precisely affects the efficiency of those laboratories that have a high test throughput.

Efforts to develop alternative and, above all faster and more efficient devices for temperature cycling, have for example led to preparing several temperature units and moving samples around between temperature units. So, for example, WO 90/05023 discloses a device to select the temperature settings for a sample at various values including a sample reception block with high thermal conductivity and a facility to set the temperature using at least two thermostatic bodies. The sample reception block is brought into contact with the body using a transport device. The movement of the sample reception block takes place by means of displacement, so that in a specified time each sample is set at a stated temperature.

Additionally, WO 2008/034896 A2 provides a device for performing a real-time PCR, in which each sample can be put in contact with various temperature areas in sequence, and in which the test vessel, that is, the reaction chamber, is moved in steps by a stepping motor from one temperature area to another temperature area.

In addition US 2002/0110899 A1 describes a “rotation thermo cycler” with several stations for receiving test vessels, in which the stations are designed to be set at various temperatures. Samples can be moved step by step from one station to the next by means of moving the test vessel. Thus, each sample can be set one after another at various temperatures.

U.S. Pat. No. 6,875,602 B2 shows a portable thermo cycler, in which several heating blocks are arranged on a rotating plate. The sample vessels can be built in the form of capillary tubes placed in cartridges and are moved step by step to the heating blocks. The capillary tubes including the samples are moved from one temperature area to another temperature area by moving the cartridges and thus are exposed sequentially to the various temperatures.

However in all the stated devices for performing PCR, the temperature setting of samples from one temperature value to the next is quicker than in conventional devices, in which repeated heating and cooling of a single temperature unit is required. But, the problem still arises that the thermal transmission of the temperature units to the test vessel and thus, to the sample is not optimal. Mostly, through small contact surfaces between the test vessel and the temperature unit, for example in flat bottomed vessels, long incubation times are required to guarantee the temperature transfer to the sample, through which the required temperature is achieved at each point of the test volume and controlled for the duration of the required reaction time. Lastly, the devices described thus do not lead to significantly discernible time savings compared with conventional PCR devices.

In a further alternative procedure for performing PCR, the reaction mix is moved through a channel, which repeatedly crosses through various temperature areas. This kind of PCR procedure and a device to perform it is shown, e.g., in EP 1 584 692 B1. Here, discs are described in which concentric temperature areas are defined, e.g. by the means of two infrared ring heating devices. A channel crosses these temperature areas in zigzag shape. The reaction mix is moved along the channel by means of centrifugal force and thus passes through the various temperature areas.

The disadvantage of the device described in EP 1 584 692 B1 is that the flow rate of the reaction mix, and thus the period of stay in a single temperature area, is set by the rotation speed of the disc. This flow rate can vary greatly, however, according to the viscosity of individual samples, through which reasonable and uniform thermal cycling must be re-set for each individual sample. The disc described thus displays a very complex and thus expensive expendable material.

Lastly, in DE 694 29 038 T2 a device for multiplying nucleic acid is set out, which contains a capillary tube reaction chamber, a primary and secondary heating device and a positioning device. Contact is made between the sample and the various temperature areas amongst other things, by means of “pumping through” the sample through the capillary tubes or through moving the heating device. Both pumping the sample through the capillary tubes and the movement of the heating device is intricate and requires a relatively high technical effort.

### SUMMARY OF THE INVENTION

It is the object of the present invention to provide a device for performing a PCR which enables very rapid thermal cycling, which is also simple to handle and also affordable and universally deployable.

This object is solved according to the invention, with the device in accordance with independent claim 1. Further beneficial details, aspects and embodiments of the present invention are stated in the dependent claims, the description, the figures and the examples.

In the context of the present invention, the following abbreviations and notions will be used:
PCR Polymerase Chain Reaction
A Adenine
G Guanine
C Cytosine
T Thymine
U Uridine
Base A, G, T, C or U
bp Base pair
dNTP Deoxy-ribonucleoside triphosphate; a mix of dATP (Deoxy-adenosine triphosphate), dCTP (Deoxy-cytosine triphosphate), dGTP (Deoxy-guanine triphosphate) and dTTP (Deoxy-thymine triphosphate) as building blocks for DNA synthesis; equivalent to nucleotide
Nucleic acid oligomer Nucleic acid of a base length that is not further specified, (e.g. Nucleic acid Octamer: a nucleic acid with the required backbone, in which eight pyrimidine or purine bases are covalently bonded to each other).
ns-Oligomer Nucleic acid oligomer
Oligomer Equivalent to nucleic acid oligomer.
Oligonucleotide Equivalent to oligomer or nucleic acid oligomer, such as e.g. a DNA-, PNA- or RNA fragment of a base length that is not further specified
Oligo Abbreviation for oligo nucleotide.
Base runs Equivalent to sequence
Nucleotide Equivalent to dNTP.
Sequence Precise series of individual bases A, C, G, T (or U) within a nucleic acid molecule.
Nucleic acid At least two covalently linked nucleotides or at least two covalently linked pyrimidine (e.g., cytosine, thymine or uracil) or purine bases (e.g., adenine or guanine). The term nucleic acid refers to any backbone of the covalently joined pyrimidine or purine bases, such as the sugar-phosphate backbone of DNA, cDNA or RNA, a peptide backbone of PNA, or analogous structures (e.g. a phosphoramide, thiophosphate or dithiophosphate backbone). An essential feature of a nucleic acid according to the present invention is that it can sequence-specifically bind naturally occurring cDNA or RNA.
Matrix A ‘template’ nucleic acid molecule; equivalent to sample, sample nucleic acid, sample DNA or template.
DNA Deoxyribonucleic acid
RNA Ribonucleic acid
Template Sample nucleic acid, which is amplified by means of PCR; equivalent to matrix
Primer Short, single stranded oligonucleotide with a base sequence complementary to a section of the sample nucleic acid, which is used as the starting point for the synthesis; the primer binds in a so-called hybridizing step (also known as: annealing) to complementary sections in single strands of the existing test nucleic acid, whereby a short double-stranded DNA section is made available, to which the polymerase attaches and synthesizes the second DNA strand by adding further complementary nucleotides.
Polymerase An enzyme, which synthesizes double stranded nucleic acid molecules, using an existing single strand as a template and assembling the nucleotides for the complementary strand according to the sequence of the template.
In the PCR method a thermo-stable polymerase, e.g. Taq-Polymerase (which was originally isolated from a thermophile organism Thermus aquaticus) is used, whose optimum working temperature lies in a range between 70° C. and 74° C. and which is also tolerant of temperatures of up to 100° C. and more.
Mismatch To form the Watson-Crick structure of double-stranded nucleic acid oligomers, the two single strands hybridize in such a way that the base A (or C) of one strand forms hydrogen bonds with the base T (or G) of the other strand (in RNA, T is replaced by uracil). Any other base pairing within the hybrid does not form hydrogen bonds, distorts the structure and is referred to as a “mismatch”.
SS Single strand
ds Double strand
Cycle Sequence of denaturation, hybridization (also known as annealing) and extension

The present invention provides a device for performing a PCR procedure, in which the device contains at least one test cell with a cavity to receive a sample and at least a first, a second and a third independently regulatable temperature unit. The three temperature units define three spatially discrete temperature areas. The device contains a means for performing a rotary movement of the test cell, in which the cavity intended to receive the test sample can be moved through the three temperature areas by means of the rotary motion of the test cell. In at least one position of the test cell, the cavity intended to receive the test sample is in contact with at least two temperature areas.

Due to the rotary movement of the test cell and the associated movement through the three temperature areas of the test sample contained in the cavity, the time consuming and repeated heating and cooling steps during PCR are avoided. The means of rotary power is preferably performed by an axle, which is set in rotation by an appropriate drive. The test cell is preferably fixed to the axle in such a way that the axle essentially goes vertically through the test cell. It is especially beneficial for the axle to penetrate the test cell approximately in an area around its center. For instance, using an axially symmetric arrangement of temperature units around this rotating axle, the test cell is always moved through the temperature areas using an equal driving force.

The simultaneous contact of the cavity intended for receiving the test sample with at least two temperature areas, according to the invention, works positively on the effectiveness and speed of the PCR reaction. Single sections of the cavity, each containing a part of the test sample volume, remain in contact each with one temperature area. The temperature equalization of these small partial volumes with the temperatures required to perform a PCR reaction, takes place very quickly because of the reduced volumes. In addition, various sections of the cavity can be set at different temperatures at the same time. Reducing the volumes to be heated or cooled and making the heating and cooling steps run in parallel for single partial volumes leads to time savings of up to two minutes per PCR cycle.

Because of the fast changes in temperature, undesired secondary reactions can be minimized. So, for example, continual formation of “haphazard” primer and/or template DNA hybrids occurring during slow cooling can be drastically reduced, which leads to the PCR running evenly throughout the reaction time. Similarly, due to fast temperature changes, the frequency of errors in the polymerase is reduced, since in slow temperature changes over long periods prevailing temperatures exist in which the polymerase is still active, but cannot work optimally and thus is liable to error.

In accordance with a preferred embodiment of the present invention, the cavity designed to receive the test sample is in contact with at least two temperature areas independent of the position of the test cell. Accordingly, it is guaranteed that the PCR reaction can run more effectively and faster at all times, and in all rotary positions for the test cell, because of the positive effects detailed above.

Preferably, the test cell is a disc wherein the cavity intended to receive the test sample extends in a circular arc with a central angle of 180° and a circular arc with a central angle of close to 360°. Embodiments in which the cavity intended to receive the test sample extends over a circular arc with a central angle of about 225° or about 270° are also preferred. As long as it is ensured that the cavity in at least one rotary position is in contact with at least two temperature areas and depending on the extension and the relative order of the temperature areas, embodiments are possible, in which a circular arc with a central angle of about 45°, about 90° or about 135° may be envisioned. Due to the flat base shape of the disc and the cavity arranged in the disc, for example by a cutting procedure, e.g., by milling, the upper surface/volume ratio is set for optimal thermal transmission in the shortest time.

In embodiments where the cavity of the test cell designed to receive the test sample extends over a circular arc with a central angle of approximately 360, the cavity shows especially advantageously, in essence the form of a hollow cylinder. By the expression “in essence showing the shape of a hollow cylinder” it should be understood that the form of the cavity designed to receive the test sample deviates from an ideal hollow cylinder in that at least one, but as a rule two openings for filling in the test sample are present. The cavity thus has two ends and is not closed to form an ideal hollow cylinder. In embodiments where the cavity extends over a circular arc with a central angle of less than 360°, the cavity takes the corresponding favored shape of a partial hollow cylinder.

By means of selecting a hollow cylinder and partial hollow cylinders, a good surface to volume ratio is achieved. In this, the radius of the hollow cylinder is selected in such a way that the hollow cylinder can contain the reaction volume needed for PCR. As a rule the hollow cylinder ends in two openings, which are used for filling in the test samples. Through filling the test sample through one opening the air contained in the hollow cylinder can escape through the second opening and the test sample is thus evenly distributed. After filling, the openings can be closed and made watertight using special stoppers or using laboratory grease and cyanoacrylate.

Embodiments in which several hollow cylinders are set concentrically in the test cell or several parts of a hollow cylinder are set concentrically and/or in a circular line in the test cell, are especially beneficial. In some embodiments, several PCR applications can run in parallel using a single test cell.

In accordance with an especially preferred embodiment of the present invention, the test cell is made of two parts, with one part built as a retaining device for a capillary and the second part built as a capillary formed to receive a test sample, in which the capillary is connected to the retaining device by clamp seating. The retaining device is especially beneficial formed in essence as a disc with an upper side, a lower side and a disc edge and is provided with a recess for receiving the capillary on its upper side, its lower side or the disc edge. Special advantages arise if the recess is circulating, since in this case it offers the possibility of clamping in one longer capillary or two or more shorter capillaries in any desired order, through which several PCR applications can be performed in parallel by means of a single test cell.

In the most advantageous case, the capillary will be fastened by clamp seating in the recess of the retaining device, so that in the end at most half of the circumference of the capillary wall is taken up by the recess. In this way it is guaranteed that at least half of the circumference of the capillary wall is in the plane with the surface of the disc on its upper side, lower side or on the disc edge or overhangs the surface, thus ensuring sufficient contact to the temperature units and an optimum thermal transmission.

The capillaries fastened to the retaining device are made, for example, from polypropylene, from polycarbonate or from Teflon and can, for example, be closed in a liquid-tight way after filling with test samples by melting either end using a small flame.

Advantageously the first, second and third temperature units include at least one temperature settable block, The blocks, wherein the temperature may be set, are made from a material with good thermal conductance, preferably of metal, wherein aluminum is especially suitable. These blocks can be brought up to the required temperatures by means of an appropriate component for beneficial energy transmission, for example by means of a heating mat or a Peltier element and by means of an appropriate temperate measurement component, e.g., by means of a resistor circuit board using appropriate regulation electronics (e.g. a PID regulator).

The temperature areas defined by means of the temperature units can be extended or expanded with appropriate insulating materials. So for example, a large size hollow body in expanded polystyrene (Styrofoam) can be placed over one, two, or all three temperature regulating blocks, through which the desired temperature in virtually all the hollow body space is set by means of the temperature regulating blocks. Thus the temperature area can be extended. At the same time, the individual temperature areas are insulated from each other. Besides Styrofoam, quite a number of other insulating materials from other known thermally resisting substances are suitable such as, for example, mineral fibers in the form of rock wool or fiberglass, wood wool, hemp, felt or cork.

In accordance with a particularly preferred embodiment of the device of the invention, the temperature settable blocks of the first, second or third temperature units, respectively, at least partially indicate the intake of the test cell by means of a notch. Through this notch at least the edge area of the test cell is taken into the temperature blocks and the cavity with the test sample is slid in, between the blocks. In an especially preferred embodiment, the cavity is arranged at the edge area of the test cell in a way that the wall surrounding the cavity has a thickness of less than 500 μm on the upper side of the preferably disc shaped test cell, as well as on the lower side and the disc edge. In this way, the test sample remains in close contact on three sides with the relevant temperature block, which leads to faster temperature changes in the sample located in the cavity.

In an especially preferred embodiment, the gap width of the notch can be varied using an appropriate shifting unit such as, e.g., varying elongated holes, and by means of this the contact between the test cell and the block can be set at the optimum. Accordingly, good thermal transmission is guaranteed, and in addition other various test cells having different dimensions and geometries can be applied. The addition of lubricants, such as mineral oil enables the friction-free movement of the test cell and leads thus to improved thermal transmission.

It is especially preferred if the test cell wall facing a temperature unit has a thickness of less than 500 μm at least in the region of the cavity intended for test sample intake. It is more preferred if the thickness is less than 300 μm and even more preferred if it is less than 200 μm. The thickness of the test cell wall influences the rate of thermal transmission, which means the thinner the wall, the faster the heat from the temperature units will be transmitted into the test sample. At the same time, the wall thickness cannot be reduced at will for stability reasons. Both thermal transmission and stability depend on the materials used, and thus the wall thickness must be established in accordance with the materials and geometry of the test cell

Preferably, the test cell shows a diameter of between 10 mm and 50 mm, and especially preferred embodiments show a diameter of between 20 mm and 30 mm. The thickness of the test cell is preferably between 0.2 mm and 1.5 mm, and especially preferred at around 1 mm. The cavity intended to receive the test sample would have a preferred depth of 0.1 mm to 0.8 mm, and a depth of 0.5 mm is especially preferred, and ends in two openings. The cavity is preferred with a volume of between 1 μl and 50 μl, and especially preferred with a volume of 20 μl, so it can take in the required reaction volume for a PCR procedure. For an especially preferred embodiment of the device in accordance with the invention, the cavity intended to receive the test sample in the test cell is fitted with a sensor to detect nucleic acid oligomer hybridizing events. This is especially beneficial with a surface sensitive sensor to detect nucleic acid oligomer hybridizing events. In essence, the sensor is formed from a modified surface, in which the modification consists in the attachment of at least one kind of probe for nucleic acid oligomers. The notion of “surface” refers to any supporting material, which is suited for immediate covalent bonding, or bonding due to other specific interactions or bonding after appropriate chemical modification of derivative or non-derivative probe nucleic acid oligomers. The solid support can be made of conductive or of non-conductive materials. Methods for immobilizing nucleic acid oligomers at the surface are known to one skilled in the art.

Preferably, the sensor is adapted and designed for spectroscopic, electrochemical or electrochemically luminescent detection of nucleic acid oligomer hybridizing events. Surface sensitive detection enables the exclusive detection of signal nucleic acid oligomers bound to the surface.

Especially preferred, the sensor is a DNA chip adapted and designed for the electrochemical detection of nucleic acid oligomer hybridizing events. In this case the modified surface exhibits at least 2 essentially spatially discrete areas, and it is preferred if there are at least 4 of these and especially preferred if there are at least 12 essentially spatially discrete areas. It is particularly preferred if the modified surface exhibits at least 32 and in particular at least 64, and especially preferably at least 96 essentially spatially discrete areas.

By “essentially spatially discrete areas”, of the surface it is meant, areas which are modified by binding of predominantly one specific kind of nucleic acid oligomer probes. Only in areas in which two such essentially spatially discrete areas adjoin one another, can a mixing of different kinds of nucleic acid oligomer probes occur.

A special advantage arises if additionally a fourth temperature unit is provided, wherein the fourth temperature unit defines a fourth temperature area spatially discrete from the three temperature areas.

By providing a fourth temperature area that is defined by a fourth temperature unit and is spatially discrete from the three temperature areas in the device in accordance with the invention, it is possible to set the temperature in partial areas of the test cell, and in particular, the inside area of the test cell may be set at a temperature that is different from the temperatures required to perform the PCR. Through selecting the geometry of the temperature setting block of the fourth temperature unit and through choosing the position within the device, the area of the test cell which is located within the fourth temperature area can be established. Accordingly, the duration and frequency of incubation in the fourth temperature can be freely chosen by setting the rotation speed for the test cell.

The fourth temperature area is indicated as especially advantageous for detecting nucleic acid oligomer hybridizing events by means of a sensor. In this case the area of the test cell in which the sensor is located is positioned in the fourth temperature area, whereby optimum temperature conditions for detection can be created. Especially when using DNA chips as sensors, the appropriate temperature for this kind of detection can be set to around 50° C.

The present invention also covers the use of the device according to the invention for the performance of PCR and especially for the performance of a real time PCR.

If using the device of the invention to perform a PCR, an analysis device enclosed in an external detection system may be particularly preferred. A miniaturized gel electrophoresis capillary system is an example of an external detection system. In order to exclude the risk of contamination, as far as possible, during the transfer of fluids from the test cell to the gel electrophoresis capillary, it is reasonable to build an “almost enclosed” system. For this purpose the wall of the test cell preferably exhibits a circular-shaped predetermined breaking point in an area in which the cavity is formed. The predetermined breaking point is then precisely adapted to the capillary diameter, so that pressurizing the capillary to the predetermined breaking point, the test cell wall is broken through at that point and the capillary can thus penetrate in a liquid-tight way into the test cell wall and the liquid located in the cavity is sucked by capillary action into the capillary.

The device according to the invention for performing a PCR can be used in a wholly preferred manner in combination with a method for the detection of nucleic acid oligomer hybridizing events as a final point display and/or in real time. The detection of PCR products thus takes place by means of a sensor fitted for the detection of nucleic acid oligomer hybridizing events, which is provided in the test cell cavity designed for receiving the test sample. It is especially preferred if the sensor is fitted for a surface sensitive detection of nucleic acid oligomer hybridizing events.

Especially preferred the method for the detection of nucleic acid oligomer hybridizing events is an end point method for the detection of PCR products including the steps providing a modified surface, the modification consisting in attaching at least one type of probe nucleic acid oligomer, providing at least one type of signal nucleic acid oligomers, the signal nucleic acid oligomers being modified with at least one detection label and the signal nucleic acid oligomers having a section that is complementary or largely complementary to the probe nucleic acid oligomers, providing a sample having target nucleic acid oligomers, bringing a defined quantity of the signal nucleic acid oligomers into contact with the modified surface, bringing the sample and the target nucleic acid oligomers contained therein into contact with the modified surface, detecting the signal nucleic acid oligomers and comparing the values obtained when detecting the signal nucleic acid oligomers with reference values The signal nucleic acid oligomers thus contain a larger number of bases than the probe nucleic acid oligomers and exhibits at least one docking section, the docking section exhibiting no structure complementary or largely complementary to any section of the probe nucleic acid oligomers and the target nucleic acid oligomers having a section complementary or largely complementary to the docking section.

Due to the docking section an association with the target nucleic acid oligomers with the signal nucleic acid oligomers occurs at a very high rate. In other displacement assays for detecting nucleic acid oligomer hybridizing events, known in the art, probe nucleic acid oligomers and signal nucleic acid oligomers are present as a hybridized double strand when the target nucleic acid oligomers are added, or probe nucleic acid oligomers and target nucleic acid oligomers are present as a hybridized double strand when the signal nucleic acid oligomers are added, respectively. Accordingly, before binding to nucleic acid oligomer components, the binding of the hybridized double strands must be dissolved.

The nucleic acid oligomer components used in accordance with the procedure described above, i.e., the probe nucleic acid oligomers and the signal nucleic acid oligomers, have a different number of bases. The signal nucleic acid oligomers have a larger number of bases and provide a docking section, which is present in a non-hybridized state, since none of the probe nucleic acid oligomers or any of the further structures is complementary with this section.

At the same time, however, the target nucleic acid oligomers now exhibit a section that is complementary or largely complementary to the docking section. When the target nucleic acid oligomers are added, they may bind directly to this docking section without prior displacement of a hybridized component. In the course of following hybridization with signal nucleic acid oligomers, the hybridized nucleic acid oligomer components must indeed be replaced, however, due to the hybridization that already occurred with the docking section, this replacement occurs at a significantly higher speed.

A further method for the detection of nucleic acid oligomer hybridizing events, which can be performed in an especially preferred way by using the device of the invention for performing a PCR, and in particular a real time PCR, including the steps providing a modified surface, the modification consisting of attaching at least one kind of nucleic acid oligomer probe, providing a sample having target nucleic acid oligomers, providing a solution with at least one type of signal nucleic acid oligomer, the signal nucleic acid oligomer being modified with at least one detection label, and the signal nucleic acid oligomer having a section that is complementary or largely complementary to the nucleic acid oligomer probes and the signal nucleic acid oligomers having a section that is complementary or largely complementary to the target nucleic acid oligomers, mixing the solution with signal nucleic acid oligomers and the test sample with target nucleic acid oligomers, bringing the mix of signal nucleic acid oligomers and target nucleic acid oligomers into contact with the modified surface, and detecting the signal nucleic acid oligomers by a surface sensitive detection method, amplifying the target nucleic acid oligomers, detecting the signal nucleic acid oligomers by a surface sensitive method and comparing the values obtained with the two detection events of the signal nucleic acid oligomers.

A “largely complementary structure” in the context of the present invention refers to sequence sections in which a maximum of 20% base pair mismatches are formed. In the context of the present invention, a “largely complementary structure” preferably means sequence sections in which a maximum of 15% base pair mismatches are formed. Particularly preferred, a “largely complementary structure” means sequence sections in which a maximum of 10% base pair mismatches are formed and very particularly preferably a maximum of 5% base pair mismatches are formed.

The detection of the signal nucleic acid oligomers takes place in a preferred using the sensitive surface detection method, since in this case only the signal nucleic acid oligomers are detected exclusively on the surface. In this context, particularly preferred methods are spectroscopic, electrochemical and electrochemiluminescent methods. As a spectroscopic method, detection of fluorescence and especially of Total Internal Reflection Fluorescence (TIRF) of signal nucleic acid oligomers is preferred.

For electrochemical detection, preferably cyclic voltammetry, amperometry, chronocoulometry, impedance measurement or scanning electrochemical microscopy (SECM) are used.

The present invention also covers a method for performing a PCR using the device in accordance with the invention, in which the test cell is moved through the spatially discrete temperature areas by means of rotary motion. By selecting the rotation speed of the test cell and by setting it according to the geometry of the temperature setting blocks, the duration of the length of stay of the test samples in the various temperature areas and thus, the duration of the steps in a PCR cycle can be established. Since the different temperatures are already set through the temperature units, all of the parameters required for the PCR cycles can be established with a single parameter setting, namely the setting of the rotation speed of the test cell. This leads to a user-friendly application of the device, since complex and elaborate programming is no longer necessary. Preferably, the test cell is moved at a constant velocity.

An especially preferred embodiment wherein a procedure for performing PCR using the device in accordance with the invention, is provided in which the DNA chip is present in the fourth temperature area during the performance of electrochemical detection of nucleic acid oligomer hybridizing events. The suitable temperatures for electrochemical detection using DNA chips, as a rule, are different from the temperatures required for the PCR steps. Due to the presence of the fourth temperature area, PCR and detection can each run at their relevant preferred temperatures.

A special advantage also arises from the use of a device in accordance with the invention in one of the procedures described above, since the PCR reaction and e.g., the detection of DNA molecules can be performed in a single test cell and with no pipetting step for test sample transfer, that is, no “liquid handling” is necessary.

### BRIEF DESCRIPTION OF THE DRAWINGS

The invention should be more closely explained by means of implementation of the examples in connection with the figures. Specific reference is made to the fact that the invention must not be restricted to the stated examples.

They show:

FIG. 1 presents a schematic diagram of an embodiment of the device of the present invention for performing a PCR;

FIG. 2a presents a schematic representation of a test cell with four temperature units. FIG. 2b presents a schematic representation of several test cells with temperature units for the parallel execution of several separate PCR reactions;

FIG. 3 presents a perspective view of an embodiment of a test cell;

FIG. 4 presents a perspective view of a test cell with a built-in sensor;

FIG. 5a presents a top view of an embodiment of a two part test cell. FIG. 5b presents a schematic representation of a vertical section through the test cell of FIG. 5a. FIG. 5c presents a schematic representation of a vertical section through a further embodiment of a two part test cell; and

FIG. 6 presents the results of comparing a PCR in a standard PCR thermo cycler and in the device in accordance with the invention.

### DETAILED DESCRIPTION OF THE PRESENT INVENTION

### Materials and Methods

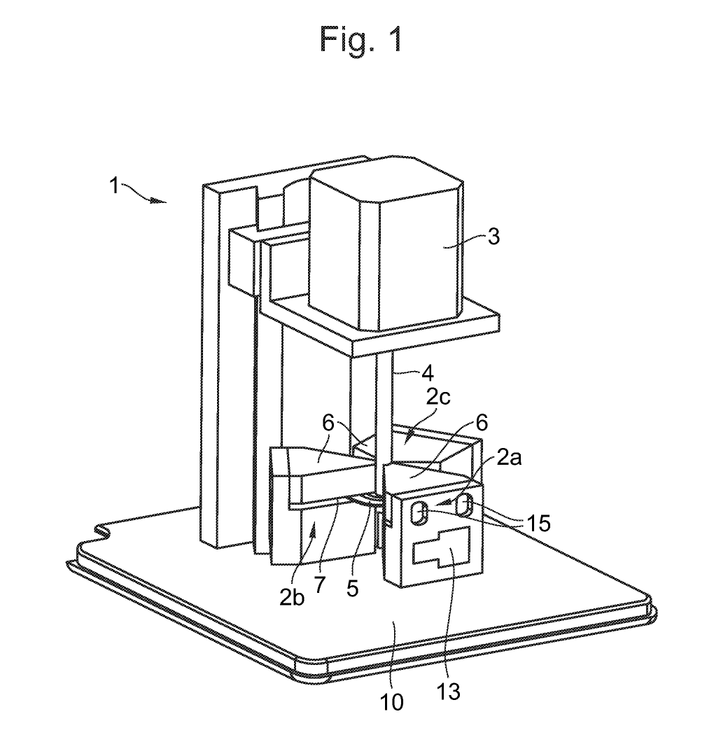

FIG. 1 shows a schematic representation of an embodiment of the device (1) for performing a PCR in accordance with the invention. In the present examples the device (1), in accordance with the invention includes a first (2a), a second (2b) and a third (2c) independently regulatable temperature unit, by which three temperature areas, in the present example 96° C., 55° C. and 72° C. are defined. The three temperature units 2a, 2b, and 2c each include a temperature settable block (6). The blocks (6) are made from a material with good thermal conductivity, and preferably Aluminum, but can also be made from other suitable metal alloys. For setting the desired temperature of each block (6) the device (1) includes a suitable means for energy conversion (13), preferable a heating mat or a Peltier element and a means for measuring temperatures, preferably a platinum resistor or a digital thermometer. By means of suitable regulatable electronics (e.g., PI or PID regulators) the three blocks (6) can be set to exact temperatures and the temperature areas can thus be defined, so that the appropriate temperatures for a PCR reaction are controlled.

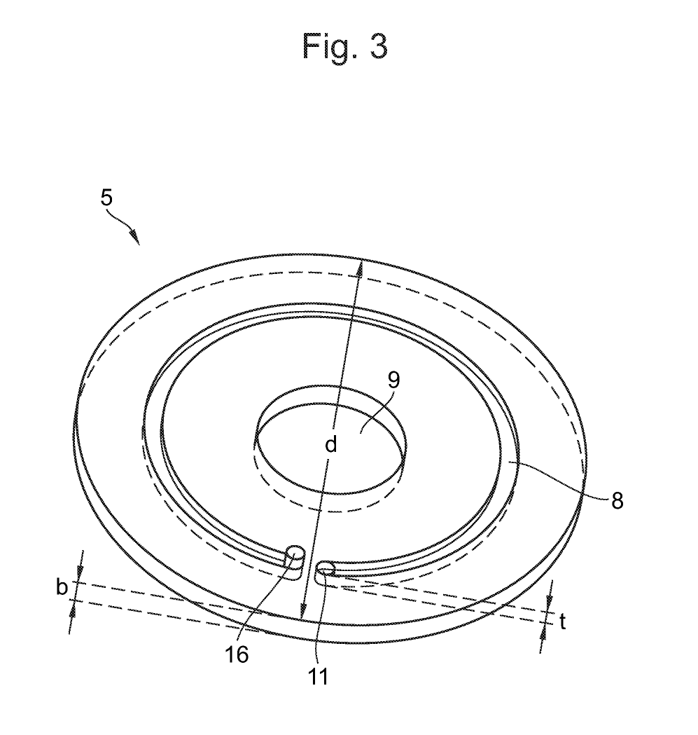

Temperature units 2a, 2b, and 2c are arranged on a base plate (10), so that in essence they form the corners of a triangle and the relative distance and/or relative position with regard to each other can be freely set. Basically, the temperature units can be set in any other desired geometry. In the middle between the temperature units 2a, 2b and 2c, the means (3), (4) for performing the rotary movement of the test cell (5) is provided. In the examples shown the means for performing the rotary motion includes an axle (4) connected to the test cell (5) and an electric motor (3). The test cell (5) of the pictured example is, as is visible for example from FIG. 3, essentially disc-shaped, in which the cavity (8) provided to receive the test sample stretches over a circular sector with a central angle of close to 360°.

Each block (6) of the three temperature units (2a, 2b and 2c) shows a notch (7), which is formed at least for partial input of the test cell (5). In the device (1), in accordance with the invention, test cell (5), which is fixed to the axle (4), is partly slid into each block (6) so that the test cell (5) is arranged with one or more partial areas in the three temperature areas and the cavity (8) is in contact with the three temperature areas, independent of the position of test cell (5).

Temperature units (2a), (2b) and (2c) show an appropriate setting unit (15), preferably with elongated holes, over which the slot width of the notch (7) can be varied and precisely adapted to the thickness of the test cell (5). Through a suitable choice of the geometry of blocks (6) and through the free settings of the distances of blocks (6) from each other the slide-in area and the slide-in depth of the test cell (5) into the individual notches (7) can be adjusted. Thus, the size and number of the partial areas of test cell (5), which are located in the individual temperature areas, can be established. In the present embodiment a 2:1 ratio of the size of the temperature areas at 72° C. to the size of the temperature areas at 96° C. and 55° C. has been selected. Between the blocks 6 an air gap is provided to prevent thermal bridging between blocks 6.

By means of the drive (3) and the axle (4), the test cell (5) is set in rotation, so that the individual partial areas of the test cell (5) are driven through the three temperature areas. The introduction of a lubricant (e.g., mineral oil) effects low friction rotation of test cell (5) and improved thermal conductivity.

By varying the speed of rotation or the rotation frequency, respectively, the time during which the critical temperatures for PCR reactions are effective at the individual partial areas of the test cell (5) can be varied.

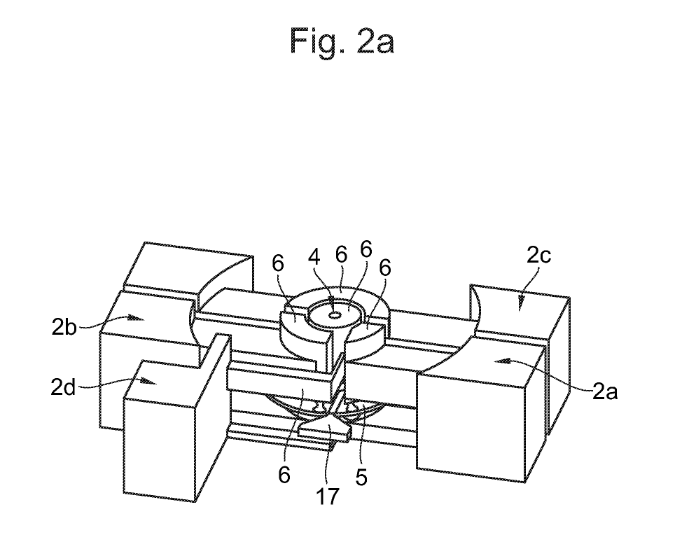

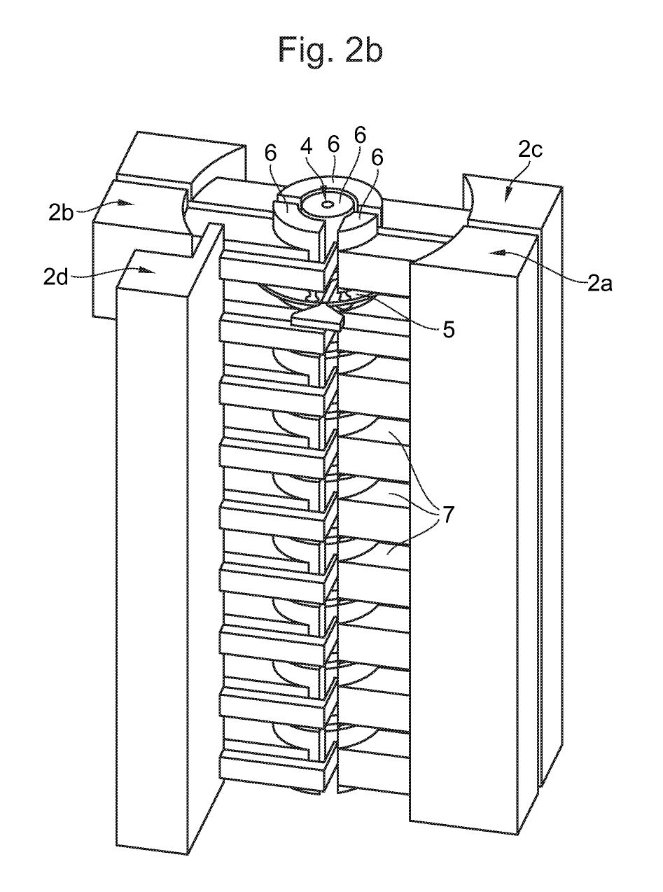

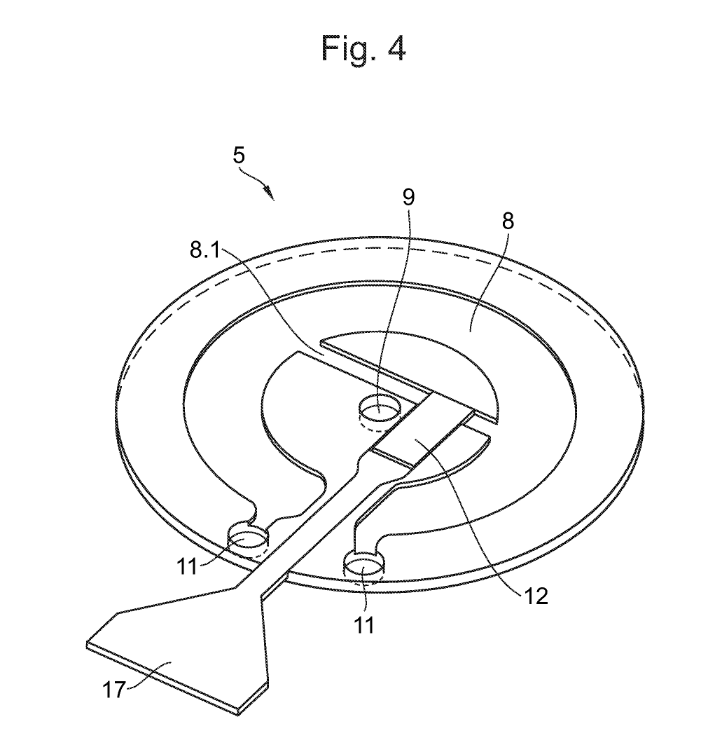

The embodiment set out in FIG. 2a provides for a fourth temperature unit (2d), through which a fourth temperature area, spatially discrete from the three temperature areas, is defined. The fourth temperature unit (2d) can hold sensors (12) contained in the test cell (5) (see FIG. 4), preferably a DNA chip at a specific temperature. A further embodiment for the device (1) with several notches (7) in each temperature settable block (6) is shown in FIG. 2b. By means of this embodiment several separate PCR reactions can be performed in a single device.

FIG. 3 shows a perspective view of an embodiment of a test cell (5). The test cell (5) shows a plane basic form, and preferably a cylindrical shape and is made from a plastic, which is temperature resistant to at least 100° C. In order to observe the sample properties it is advantageous if this is a transparent plastic.

The test cell (5) shows preferably a diameter d of between 10 mm and 50 mm and a thickness b of between 0.2 mm and 1.5 mm. The cavity (8) intended to receive the test sample extends across a circular arc with a central angle of close to 360° and is essentially made in the form of a hollow cylinder with a preferred depth t of 0.1 mm to 0.8 mm, which ends in two openings (11). The cavity (8) shows a volume of between 1 μl and 50 preferably of 20 μl, and thus is able to contain the reaction volume required for a PCR procedure.

Test cell (5) shows a drill hole (9), by means of which test cell (5) is fastened in the device (1) in accordance with the invention, by a means (3), (4) for performing the rotary movement of the test cell (5), preferably to a rotating axle (4).

To seal the test cell (5) the openings (11) in the cavity (8) can be closed using laboratory grease, e.g., Glisseal®N and cyanocarylate, or using a special plug 16, preferably made of rubber or plastic, after filling the test cell (5) with the PCR reaction mix.

FIG. 4 shows a perspective view of a further embodiment of the test cell (5) in accordance with the invention, with a built-in sensor (12). The cylindrical test cell (5) shows a cavity (8) to receive the test samples. Adjacent thereto is provided a drill hole (9), by means of which test cell (5) in the device, in accordance with the invention, is fastened to a means for performing the rotary movement of the test cell (5), preferably to a rotating axle. The sensor (12) in the present embodiment is a DNA chip. The cavity (8) containing the PCR reaction mix, essentially in the form of a hollow cylinder, is extended through a bifurcating channel (8.1), which is connected with the surface of the sensor. Through this, the test sample to be analyzed, namely the PCR reaction mix, arrives at sensor (12). In the embodiment portrayed, an electrochemical detection of PCR products is performed using the integrated sensor (12), which is connected to a PCB conductor board output.

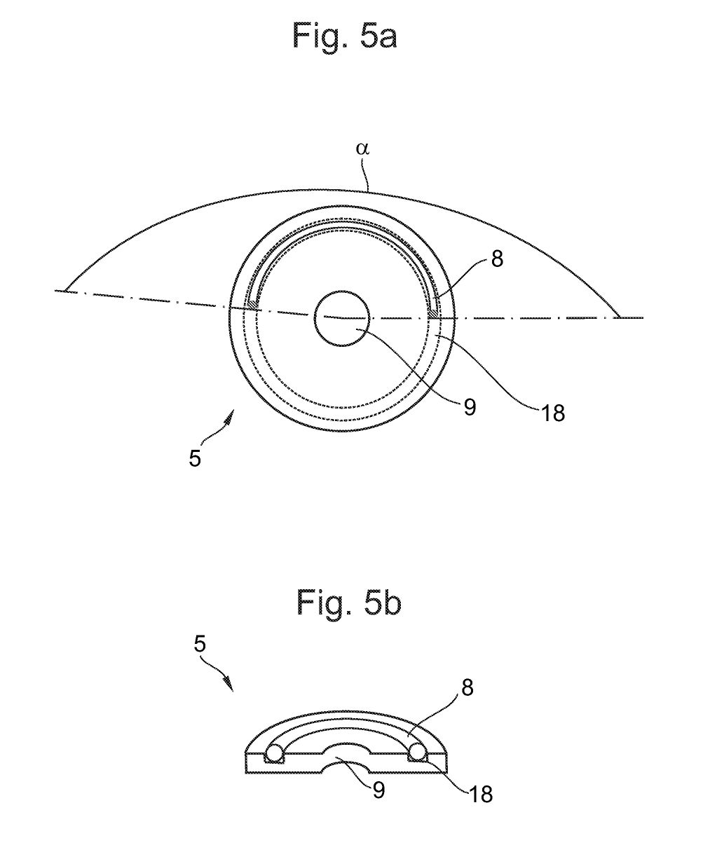

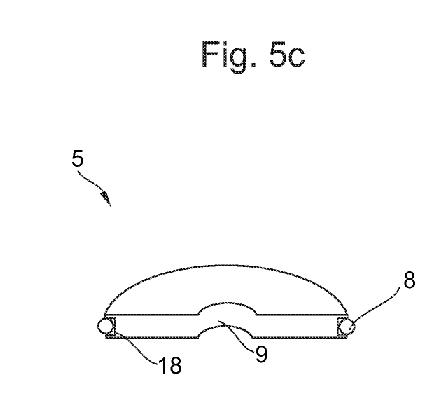

In FIGS. 5a, 5b and 5c, further embodiments of a test cell (5) with cavity (8) are shown, namely two-part embodiments are shown. In the two-part embodiments the test cell (5) is formed as a retaining device for a capillary, and the cavity (8), intended to receive the test sample, is formed by means of a capillary. The retaining device (5) shows a recess (18) to receive the capillary (8), in which the capillary (8) is clamped to a clamped-closing connection. FIG. 5a shows a top view of a two-part embodiment, whilst FIG. 5b shows a vertical section through the test cell shown in FIG. 5a in which a revolving recess (18) that receives the capillary (8) runs along the upper side of the disc-shaped retaining device (5). The capillary (8) that is clamped into the retaining device (5) extends across a circular arc with a central angle α of about 170°. FIG. 5c shows a vertical section through a further two-part embodiment, in which the revolving recess (18) that receives the capillary (8) runs around the disc edge of the retaining device (5).

### EXAMPLES

### Example 1

### Manufacturing a Test Cell

The test cell is made from two plastic parts, namely a lower part comprising the cavity intended to receive the test sample, and an upper part. Through a cutting manufacturing procedure such as milling, the lower plastic part of a test cell with a diameter of roughly 15 mm and a thickness of about 1 mm also containing the cavity, is made from a polymethylmethacrylate rough item, in which a 0.5 mm deep cavity (8) in the form of a hollow cylinder is milled, which ends in two pierced openings (11). In the center of the lower part of the test cell a drill hole (9) is made.

To manufacture the test cell an acrylic glass disc is glued onto the lower plastic part using a suitable adhesive. The acrylic glass disc is connected in a liquid-tight manner with the lower form-giving part of acrylic glass. Cyanoacrylate or dichloromethane can be used, for example, as an adhesive for the acrylic glass. Other polymers may require other adhesives. After filling the cell with the PCR reaction mix, the openings (11) of the cavity (8) can be closed using laboratory grease (e.g., Glisseal®N) and cyanoacrylate or by means of a suitable rubber plug.

For embodiments with improved thermal conductivity from the temperature units 2a, 2b and 2c to the test cell (5), materials with higher thermal conductivity can be used or the wall thicknesses of the materials can be reduced. Equally, other procedures for connecting the lower form-giving plastic part of test cell (5) to the upper plastic disc can be envisaged, such as welding procedures, for example, laser plastic welding.

Alternative cavity geometries with any built-in sensors (12) can be manufactured using cutting manufacturing processes, casting manufacturing procedures (e.g., injection molding technology), stereolithography or other suitable procedures. An integrated sensor (12) can, for example, be made to contact an output PCB contact board (17), as shown in FIG. 4. In addition, several separate cavities (8) with several separate openings (11) can be inserted in a test cell (5) wherein, so that several PCR reactions can run in parallel in a single test cell (5). In this case it is also possible to analyze the test sample using a common sensor or several sensors (12) (e.g., one sensor (12) per cavity (8)).

Further, alternative embodiments for a test cell (5) enable a contamination-free “quasi-enclosed” liquid transfer from the cavity (8) of the test cell (5) to an external detection system, for example, a miniaturized gel electro-phoresis capillary. For this purpose the wall of the test cell comprises an essentially circular predetermined breaking point in the area in which the cavity (8) is formed. The predetermined breaking point can be incorporated, e.g., through precise reductions in the wall thickness. By pressuring the capillary at this point, the wall of the test cell (5) is broken through and the capillary will come into connection with the cavity (8) and the test sample contained therein, which will be sucked into the capillary, by capillary action.

Embodiments are also possible in which at the predetermined breaking point an additional “female” connection side (inward cone) for a Luer lock connection is comprised. By applying the “male” opposite face (outward cone of a Luer lock connection, for example by a syringe or a cannula, the predetermined breaking point can be pierced through and a “quasi enclosed” system between the cavity of the test cell and the inner space of the syringe can be made.

### Example 2

### Performing a Comparative PCR

The PCR (polymerase chain reaction) forms a standard method in molecular biology, which was developed in 1984 by Kary Mullis. It enables amplification of (multiplying) specific DNA sequences using simple test arrangements. Hence, a certain sequence from a large gene (e.g., the code for a metabolic protein) can be isolated and multiplied. Primers are used which serve as markers limiting this specific sequence and to which the DNA-polymerase can bind.

For a standard PCR reaction, a so-called master mix is prepared. For this a reaction vessel, e.g., a 2 ml micro screw cap tube is labeled accordingly and placed in a cooling rack (0° C.-4° C.). The primers (concentration generally around 10 μM), the dNTP mix (each dNTP 25 μM; dNTP=Dioxy-ribonucleic acid triphosphate, i.e. DNA building blocks), standard PCR buffer and MgAc (100 mM) are added to the tube and thoroughly mixed.

The DNA template to be multiplied (i.e. the isolated, and purified DNA material intended for multiplication) is placed in 0.2 ml or 0.5 ml capacity PCR reaction vessels or PCR tubes (in the cooling rack at ca. 0° C.-4° C.) (as a rule 5 to 10 μl) and filled with master mix to, as a rule, 20 μl to 50 μl. As a blank test sample a PCR tube is filled with water (DNA free) instead of the DNA template and filled to the corresponding final volume with master mix.

Immediately before the start of the PCR reaction the polymerase is added to the prepared PCR tubes (with template and master mix, and with water and master mix in the case of the blank test sample) and mixed by repeatedly pipetting up and down.

In the thermo cycler the steps of denaturation, annealing (primer hybridization) and elongation are repeatedly performed. For denaturation the double strand DNA templates are heated to 94-96° C., in order to separate the strands. In the initial cycle the DNA is often heated for a longer time (initializing), to ensure that both the output DNA and the primer have completely separated from each other and only single strands remain. During primer hybridization a temperature enabling a specific binding of the primers to the DNA is set for around 30 seconds. The precise temperature is specified in accordance with the sequence and length of the primers (mostly in a temperature range of 55° C.-65° C.). The DNA polymerase fills the missing strands with free nucleotides. It starts at the 3′ end of the hybridized primers and then follows the DNA template strand. The primer is not further detached and forms the start of the new single strand. The temperature depends on the working optimum for the DNA polymerase used (as a rule ca. 68° C.-72° C.). This step lasts about 30 seconds for each 500 base pairs, but varies according to the DNA polymerase used.

For the comparison of a standard PCR as described herein, a BioRad thermo cycler (iCycler model) was used. 100 μl of a common master mix was used for all the reactions performed, as set out in Table 1 below. 20 μl of template (DNA extract from Legionella pneumophila, about 100 DNA copies/5 μl) were mixed with 80 μl of master mix, and added to Taq Polymerase (BioTaq from BIOLINE), and split into 4 parts each of 25 μl (one for the PCR in the standard PCR thermo cycler, and three for the PCR using the device in accordance with the invention). In addition a test control blank was used (5 μl water plus 20 μl of master mix with polymerase, in the standard thermo cycler). All the PCR parameters are shown in Table 1. The device used for performing the PCR and the test cell are described in the context of FIGS. 1 to 4 and in Example 1.

TABLE 1
Master mix

| | Volume [μl] |
|---|---|
| Forward Primer (10 μM) | 6.30 |
| Reverse Primer (10 μM) | 6.30 |
| PCR Buffer* | 21.00 |
| MgAc (100 mM) | 2.63 |
| DNTP Mix (25 mM) | 0.84 |
| TaqPolymerase (BioTaq) | 0.53 |
| H20 | 62.40 |
| Total | 100.00 |

PCR parameters using a standard thermo cycler

| Temperature [° C.] | Time [s] | Number of Cycles |
|---|---|---|
| 96 | 300 | 1 |
| 96 | 10 | 40 |
| 55 | 15 | 40 |
| 72 | 15 | 40 |
| 72 | 60 | 1 |
| 4 | hold | |

PCR Parameters using the device of the invention

| Block | Temperature [° C.] | Time [s] | Cycles |
|---|---|---|---|
| A) Denaturation | | | |
| A | 96 | 300 | Rotation at ca. |
| B | 96 | 300 | 5 revolutions/ |
| C | 96 | 300 | min. |
| B) PCR | | | |
| A | 96 | Dependent on | 40 |
| B | 55 | rotation speed | 40 |
| C | 72 | and block | 40 |
| | | geometry | |

*(5 x Bicine buffer, 250 mM bicine/KOH, pH 8.2; 575 mM K-acetate, 40% glycerol (v/v), Bicine = N,N-Bis(2-hydroxyethyl) Glycine)

Using the device of the invention the reaction time of the PCR can be shortened from ca. 45 min (standard PCR) to 25 min. Optimized heating block geometry and minimized wall thicknesses of the test cell should enable further reductions in reaction time for a PCR to ca. 10 min or even less.

The results of the PCR were analyzed by using agarose gel electrophoresis. Agarose gel electrophoresis is a procedure in which DNA fragments, and in the special case PCR products, can be identified by their size. DNA is thereby put in an agarose gel and placed under a voltage. Thus, shorter DNA fragments move more quickly to the plus electrical pole than longer DNA fragments. The length of the PCR products can be established by comparing with a DNA ladder, which contains DNA fragments of known sizes and which is running in the gel in parallel.

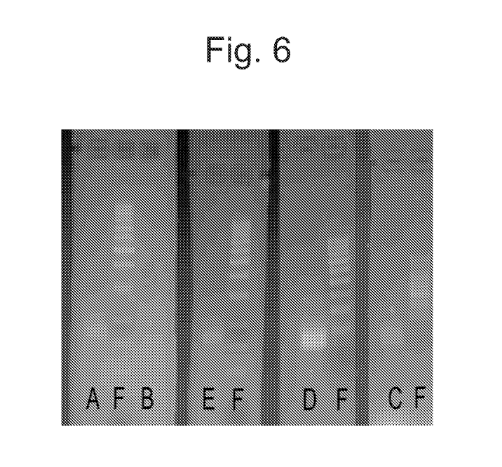

FIG. 6 shows a photograph of the PCR products analyzed by gel electrophoresis, which were obtained in the experiments set out in Table 1. In FIG. 6 a gel electrophoresis analysis for the PCR products obtained from a standard PCR reaction using a standard cycler are identified as A. The control blank performed in a standard PCR reaction in a standard cycler is identified as B. The gel electrophoresis analysis for the PCR products obtained by using the device in accordance with the invention, are shown depending on the rotation speed of the test cell, as C (0.5 revolutions per minute), D (1 revolution per minute) and E (2 revolutions per minute). F identifies a DNA ladder.

From FIG. 6 it is clearly visible that the same expected PCR product of ca. 150 base length was obtained using a standard PCR reaction in a standard cycler as was obtained in the PCR performed in the device in accordance with the invention at three different test cell rotation speeds.

### REFERENCE KEY

1 Device for performing a PCR
2a First temperature unit
2b Second temperature unit
2c Third temperature unit
2d Fourth temperature unit
3 Electric motor
4 Axis
5 Test cell
6 Settable temperature block
7 Notch
8 Cavity
8.1 Bifurcated channel
9 Drill hole
10 Floor plate
11 Openings
12 Sensor
13 Means of energy transmission
14 Means of temperature measurement
15 Setting unit
16 Stoppers
17 PCB connection board
18 Recess
d Diameter
b Test cell thickness
t Cavity depth

1. A device (1) for performing a polymerase chain reaction (PCR) comprising
at least one test cell (5) with a cavity (8) to receive a test sample;
at least a first (2a), a second (2b) and a third (2c) independently regulatable temperature units (2a, 2b, and 2c), in which the three temperature units (2a, 2b, and 2c) define three spatially discrete temperature areas; and
at least one means (3, 4) for performing a rotary movement of the test cell (5), in which the cavity (8) intended to receive the test sample can be moved through the three temperature areas by means of the rotary movement of the test cell, in which
the cavity (8) intended to receive the test sample is in contact with at least two temperature areas in at least one position of the test cell (5).

2. The device (1) according to claim 1, characterized in that the cavity (8) intended to receive the test sample is in contact with at least two temperature areas independent of the position of the test cell (5).

3. The device (1) according to claim 1, characterized in that the test cell (5) has essentially a disc shaped form.

4. The device (1) according to claim 3, characterized in that the cavity (8) of said test cell intended to receive the test sample extends in a circular arc with a central angle of at least 180°.

5. The device (1) according to claim 4, characterized in that said cavity (8) extends in a circular arc with a central angle of close to 360°.

6. The device (1) according to claim 1, characterized in that the cavity (8) in the test cell (5) intended to receive the test sample comprises the shape of a hollow cylinder.

7. The device (1) according to claim 1, characterized in that the test cell (5) is built in two parts, wherein one part is a retaining device for a capillary and a second part is a capillary for receiving the test sample, wherein said capillary is connected to said retaining device by a force-fitting means.

8. The device (I) according to claim 7, characterized in that the retaining device for the capillary comprises the shape of a disc with a lower side, an upper side, and a disc edge, wherein a recess to receive the capillary is provided on the upper side and/or the lower side and/or on the disc edge.

9. The device (1) according to claim 1, characterized in that the first (2a), the second (2b) and the third (2b) temperature units each include at least one temperature setting block (6).

10. The device (1) according to claim 9, characterized in that the temperature setting blocks (6) of the first (2a), the second (2b) and the third (2b) temperature units each comprises a notch (7) for at least partial reception of a test cell (5).

11. The device (1) according to claim 1, characterized in that a wall of the test cell (5), at least in the area of the cavity (8) intended to receive the test sample and facing the temperature units (2a, 2b, and 2c), has a thickness of less than 500 μm.

12. The device (1) according to claim 1, characterized in that the cavity (8) of the test cell (5) intended to receive the test sample comprises a sensor (12) for detection of nucleic acid oligomer hybridizing events.

13. The device (1) according to claim 12, characterized in that the sensor (12) is a sensor for spectroscopic, electrochemical or electro-chemiluminescent detection of nucleic acid oligomer hybridizing events.

14. The device (1) according to claim 12, characterized in that the sensor (12) is a DNA-chip for electro-chemical detection of nucleic acid oligomer hybridizing events.

15. The device (1) according to claim 1, further comprising a fourth temperature unit (2d) wherein the fourth temperature unit (2d) defines a fourth spatially discrete temperature area.

16. A method of using a device (1) of claim 1 for performing polymerase chain reactions (PCR) comprising moving a test cell (5) of said device (1) at a constant rotation speed through at least three spatially discrete temperature areas defined by the at least three temperature units (2a, 2b and 2c) of the device (1) of claim 1.

17. (canceled)

18. The method according to claim 16, further comprising moving said test cell (5) through a fourth discrete temperature area defined by a fourth temperature unit (2d), wherein said test cell (5) comprises a DNA chip sensor for electro-chemical detection of nucleic acid oligomer hybridizing events, and wherein said electro-chemical detection of nucleic acid oligomer hybridizing events occurs when said sensor is located within said discrete fourth temperature area.

19. The device (1) according to claim 12, wherein said sensor (12) is a sensor for detecting surface sensitive nucleic acid oligomer hybridizing events.

20. The method according to claim 16, wherein said PCR comprises a real-time PCR.

21. The method according to claim 16, wherein said rotation speed is set according to the geometry of the discrete temperature area of a device (1) and the duration of the length of stay of the test samples within the various temperature areas.
